(19) United States
(12) Patent Application Publication
(10) Pub. No.: US 2007/0233069 A1
(43) Pub. Date: Oct. 4, 2007

(54) Implant/support member interconnection mechanism

(76) Inventors: Stewart Young, Memphis, TN (US); Michael C. Sherman, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/362,958

(22) Filed: Feb. 27, 2006

(51) Int. Cl. A61F 2/30

(52) U.S. Cl. 606/61

(57) ABSTRACT

Mechanisms for connecting an elongated member for orthopedic support or therapy to bone or other tissue are disclosed. Embodiments may include a bone connecting member with a channel for accommodating an elongated member such as a spinal rod, a cap member that connects to the bone connecting member in a direction other than along the channel, for example perpendicular to the channel, and a lock member for locking the bone connecting member, cap member and elongated member together. Methods for using such embodiments are also disclosed.

The present disclosure generally concerns mechanisms used to connect orthopedic implants with elongated members, such as rods, for therapeutic or corrective purposes.

### BACKGROUND

In the field of orthopedic surgery, several types of apparatus are known for correction, support or other treatment of tissues. For example, in the spinal field, elongated support members such as rods, bars or plates are connected to vertebrae or adjacent tissue so as to provide a corrected spinal curvature, for support of injured or surgically-treated vertebrae or vertebral joints, and for other purposes. Such elongated members can be connected to bone, for example, via a variety of implants including screws, bolts, clamps, wires, hooks.

In some cases, the surgical therapy, the size of the patient and/or the anatomy to be operated on may require close placement of such implants to each other as they hold the elongated member. For example, in cervical spinal cases, or in cases involving children, placement of an elongated member for orthopedic therapy may require a number of implants very close to or touching each other in order to connect the elongated member to the proper tissue. Placement of such implants, connection of an elongated member to them, and locking the elongated member to the implants can be difficult in such cases.

### BRIEF DESCRIPTION OF THE DRAWINGS

FIG. 1 is an exploded perspective view of an embodiment of an implant.

FIG. 2 is a side partially-exploded view of the embodiment of FIG. 1.

FIG. 3 is an exploded view of an embodiment similar to that of FIG. 1 with a different connection.

FIG. 4 is an exploded perspective view of another embodiment of an implant.

FIG. 5 is side partially-exploded view of the embodiment of FIG. 4.

FIG. 6 is an exploded view of an embodiment similar to that of FIG. 1 with a different connection.

FIG. 7 is a partially-exploded perspective view of another embodiment of an implant.

FIG. 8 is a partially-exploded side view of the embodiment of FIG. 7.

FIG. 9 is a partially-exploded side view of aspects of the embodiment of FIG. 7.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated devices, and such further applications of the principles of the disclosure as illustrated therein, being contemplated as would normally occur to one skilled in the art to which the disclosure relates.

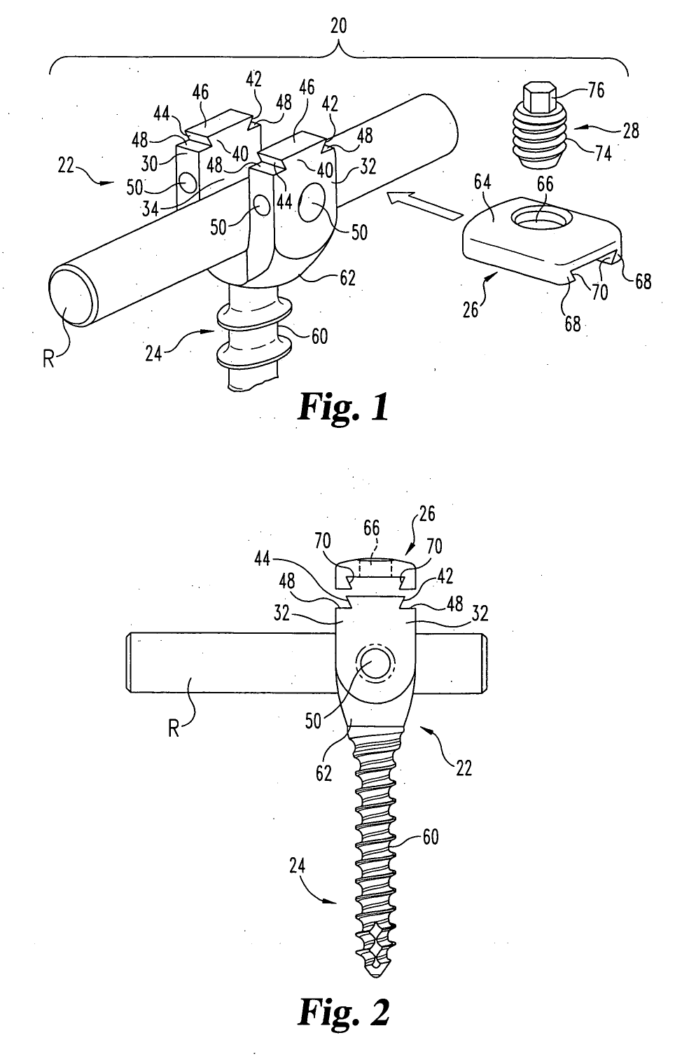

Referring now generally to FIG. 1, an embodiment of an implant 20 is shown. In that embodiment, implant 20 includes a head portion 22, a bone connecting portion 24, a cap member 26, and a lock member 28. In general, implant 20 is connected to bone and to a support member R, such as a rod, bar or other elongated member, so that the support member can provide correction, support or other benefit to an orthopedic surgical site.

Head portion 22 of implant 20 includes two branches 30, 32 that at least partially define a channel 34 for accommodating an elongated member such as an orthopedic rod. Channel 34 has a longitudinal axis substantially along or parallel to which an elongated member can lie. The illustrated embodiment of head portion 22 shows branches 30, 32 extending generally upward or away from connecting portion 24, but in other embodiments branches 30, 32 could be otherwise oriented, e.g. oblique to an axis of connecting portion 24 or substantially perpendicular to an axis of connecting portion 24, making a channel that opens somewhat or entirely to the side of such an axis. Branches 30, 32 are also shown generally parallel to each other and somewhat planar, and in other embodiments could be non-parallel with each other and/or curved.

Branches 30, 32 are substantially identical in the illustrated embodiment, each having an projection 40 on a top or outer portion, which is shaped substantially like a trapezoid or tenon. Side or lateral surfaces 42, 44 of projection 40 are sloped inwardly, in a particular embodiment, from an outer surface 46 of projection 40 toward connecting portion 24. Surfaces 42 and/or 44 may also be otherwise configured, for example to form a generally inverted T-shape. Generally flat, horizontal surfaces 48 are adjacent surfaces 42, 44 in this embodiment. On an outside surface of branches 30, 32 at least one indentation 50 is provided for accommodating a part of a holding or manipulating tool (not shown). Indentation(s) 50 may be provided on the exterior of one or both sides of one or both branches 30, 32, and/or may be placed on the surfaces immediately adjacent one or both sides of channel 34. In other embodiments, a boss or stop may be provided on one or both branches 30, 32 and/or cap member 26 for limiting movement of cap member 26 with respect to head 22, as further described below.

Connecting portion 24 is a threaded shaft 60 that is integral with or fixedly attached to head 22 in this embodiment. The threads of shaft 60 may be configured for good purchase in cancellous or other types of bone, and may be cannulated or include holes through shaft 60 perpendicular or oblique to its axis for bone ingrowth. An unthreaded convex or substantially conical joining section 62 is provided in a particular embodiment between connecting portion 24 and shaft 60. In other embodiments, connecting portion 24 may be pivotably or multi-axially connected to head 22, making implant 20 a pivotable or multi-axial screw. Types of such screws are shown in U.S. Pat. Nos. 6,280,445 and 5,797,911, both of which are incorporated by reference herein in their entireties. In yet other embodiments, connecting portion 24 may be or include a hook portion, a clamp, or other structure for connecting to tissue such as bone.

Cap member 26, in the embodiment of FIG. 1, includes a base 64, a hole 66 through base 64, and extension portions or legs 68 generally perpendicular to base 64 and on either side of hole 66. In that embodiment, legs 68 extend along substantially the entire width of base 64, although in other embodiments legs 68 could have one or more gaps between portions at or adjacent sides of base 64. Legs 68 include internal surfaces 70 that in a particular embodiment are sloped inwardly from base 64 toward the bottom of legs 68, and could be considered a mortise. For example, surfaces 70 (and surfaces 42, 44 of head 22 as well) may be configured so that substantially all of surfaces 70 are adjacent to or abut surfaces 42, 44, or may be configured so that a portion of surfaces 70 are adjacent to or abut surfaces 42, 44. In one particular embodiment the angle of surfaces 70 to base 64 is about the same as the angle of one or both of surfaces 42, 44 to upper surface 46 of projection 40. Hole 66 is threaded in one embodiment, and is positioned and of a diameter such that an extension of hole 66 would not intersect legs 68.

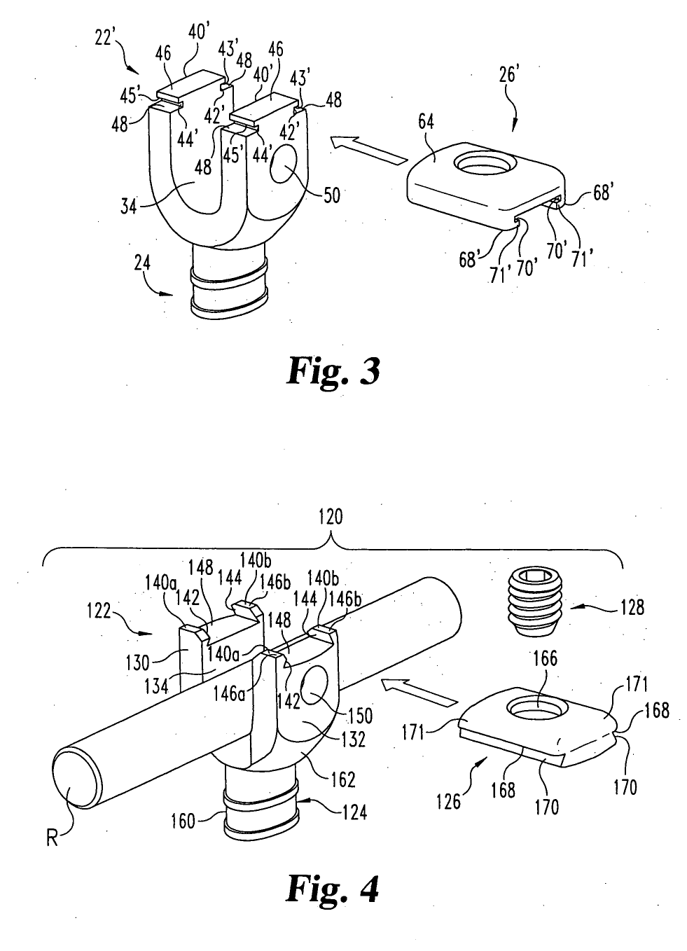

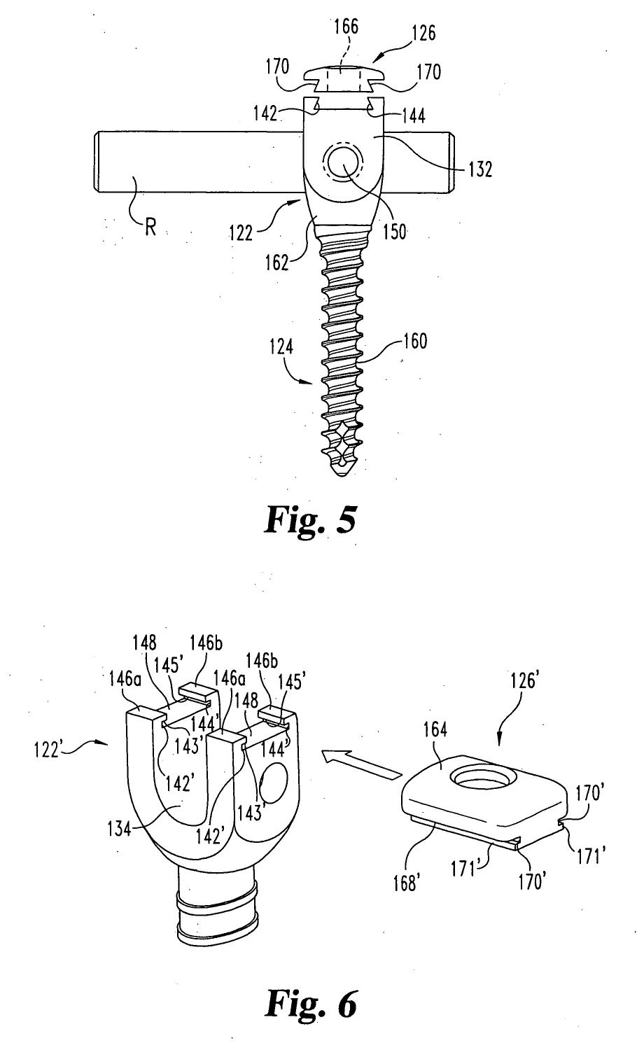

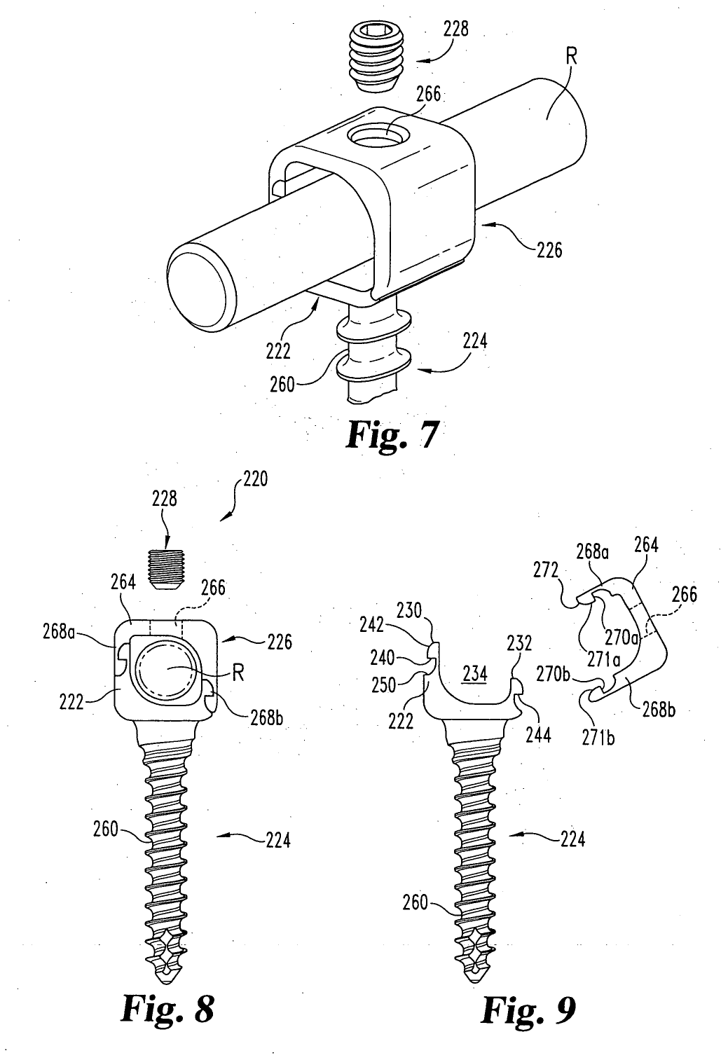

Referring to FIG. 3, another embodiment of cap member 26′ and head 22′ is shown. In most respects, cap member 26′ and head 22′ are the same as the above-discussed embodiments of cap member 26 and head 22, and for clarity's and brevity's sake similar parts are not further discussed here. The principal difference between these embodiments is the T-shape of the space in cap member 26′ and of the projection 40′ atop each branch 30, 32. Accordingly, the embodiment of head 22′ shown in FIG. 3 includes surfaces 42′, 44′ that are substantially perpendicular to surfaces 46 and 48 (and/or substantially perpendicular to channel 34), and surfaces 43′ and 45′ that are substantially parallel to surfaces 46 and 48. The embodiment of cap member 26′ includes legs 68′ with internal surfaces 70′ that are substantially perpendicular to base 64, and a flange 71′ that is substantially parallel to base 64. In addition to substantially tenon-and-mortise and T-shaped configurations, other configurations for cap member 26 and head 22 can be used.

The illustrated embodiment of lock member 28 is a set screw having a threaded lower portion 74 and an upper driving portion 76. Some embodiments of set screws may be torque-limiting, so that upper portion 76 separates from lower portion 74 on application of sufficient torque. Driving portion 76 may have an external print, such as a hexagonal formation as shown in FIG. 1, or may have an internal print such as a hexagonal or hexalobed opening, or both internal and external prints. In other embodiments, lock member 28 could be a clamp, spring-loaded, ratcheting, cam or other type of member that connects with embodiments of a cap member such as cap members 26 and 26′.

In use, implant 20 is introduced into a surgical site and connected to tissue. In the following discussion, spinal surgery will be described, although similar orthopedic surgical steps could be taken at other surgical sites. With the illustrated embodiment, a hole is drilled into a bone (e.g. a vertebra), which may then be tapped. Connecting member 24 is threaded into the hole to a desired depth and/or to a desired orientation of head 22. In many instances of spinal surgery, a surgeon will orient head 22 so that channel 34 is substantially parallel to a portion of the spine. When head 22 and connecting member 24 are placed as the surgeon desires, an elongated member (not shown) is placed at least partially in channel 34. The elongated member can be pre-bent to conform to a particular spinal curvature or as a particular correction, support or therapy requirement may dictate, or the elongated member can be bent in situ.

Once at least a portion of an elongated member is in channel 34, cap member 26 is placed on head 22. In the illustrated embodiment of implant 20, cap member 26 slides onto head 22 in a direction substantially perpendicular to channel 34 and to the longitudinal axis of connecting portion 24 (e.g. substantially along the arrow in FIG. 1). In other embodiments, cap member 26 and head 22 can be configured so that cap member 26 moves onto head 22 in other directions that are non-parallel to or substantially not along the axis of channel 34. Legs 68 of cap member 26 are positioned so that their surfaces 70 are adjacent surfaces 42, 44 of extensions 40 and legs 68 are adjacent generally horizontal surfaces 48. Cap member 26 slides across head 22, with surfaces 70 and surfaces 42, 44 sliding along each other. Cap member 26 is advanced across head 22, in the illustrated embodiment, until surfaces 70 of legs 68 are adjacent to or abut surfaces 42, 44 of both branches 30, 32. If a stop is provided, cap member 26 may be advanced across head 22 until the stop prevents further advancement of cap member 26.

When cap member 26 is in place as indicated, legs 68 and extensions 40 form essentially mortise-and-tenon or dovetail joints on each side of head 22. With cap member 26 atop head 22 so as to close head 22, the elongated member (in channel 34) and/or implant 20 can be adjusted relative to each other or to the adjacent vertebrae. For example, implant 20 can be pushed, pulled or rotated along with its attached vertebra so as to compress, distract or rotate the vertebra with respect to other bones or tissue. As another example, the elongated member can be further bent or turned in channel 34 to a better position or configuration, while cap member 26 retains the elongated member within channel 34. In embodiments in which implant 20 is a pivotable or multi-axial implant, the head of such an implant can be pivoted with respect to its bone connecting portion and/or the elongated member in the head for better support.

Once any such adjustments are made, lock member 28 is engaged to lock the elongated member to head 22 of implant 20. In the illustrated embodiment, in which lock member 28 is a set screw, lock member 28 can be threaded into hole 66 of cap member 26 prior to or after placement of cap member 26 on head 22. In either case, when implant 20 and its associated vertebra is positioned as the surgeon desires with respect to other vertebrae, implants and/or the elongated member, the set screw is tightened into contact with the elongated member to the desired degree. Lock member 28 exerts force on the elongated member, which presses back on lock member 28 and the threads of hole 66, and surfaces 70 of cap member 26 are forced against surfaces 42, 44 of head 22. The elongated member and head 22 are thus locked with respect to each other.

The embodiment shown in FIG. 3 is used in essentially the same fashion as the embodiment shown in FIG. 1. When cap member 26′ is slid onto head 22′, a square joint is formed in which flanges 71′ are adjacent surfaces 44′ and 45′. Insertion of a lock member, such as lock member 28, through cap member 26′ to exert force on an elongated member locks the construct.

Referring now generally to FIG. 4, there is shown an embodiment of an implant 120 similar to that of implant 20. Implant 120 includes a head portion 122, a bone connecting portion 124, a cap member 126, and a lock member 128. In general, the illustrated embodiment of connecting portion 124 is essentially identical to the embodiment of connecting portion 24 disclosed and/or shown above.

Head portion 122 of implant 120, like head 22 of implant 20, includes two branches 130, 132 that at least partially define a channel 134 for accommodating an elongated member R such as an orthopedic rod. Channel 134 has a longitudinal axis substantially along or parallel to which rod R can lie. The illustrated embodiment of head portion 122 shows branches 130, 132 extending generally upward or away from connecting portion 124, but in other embodiments branches 130, 132 could be otherwise oriented, e.g. oblique to an axis of connecting portion 124 or substantially perpendicular to an axis of connecting portion 124, making a channel that opens somewhat or entirely to the side of such an axis. Branches 130, 132 are also shown generally parallel to each other, and in other embodiments could be non-parallel with each other.

Branches 130, 132 are substantially identical in the illustrated embodiment, each having a pair of projections 140a, 140b on a top or outer portion. Side or lateral surface 142 of projection 140a and side or lateral surface 144 of projection 140b are sloped outwardly, in a particular embodiment, from an outer surface 146a, 146b of extensions 140a, 140b toward connecting portion 124. Surfaces 142 and 144 may be thought of as undercuts or undercut surfaces, and may form a mortise-type opening, a generally inverted T-shaped opening, or have another appropriate configuration. A generally flat, horizontal surface 148 is adjacent surfaces 142, 144 in this embodiment. Another embodiment of implant 120 could include only projection 140a on branch 130 and projection 140b on branch 132, or could include two projections on one branch and one on the other. On an outside surface of branches 130, 132 an indentation 150 is provided for accommodating a part of a holding or manipulating tool (not shown). As already noted, connecting portion 124 is essentially identical to the embodiment of connecting portion 24 above, and thus includes a threaded shaft 160 that is integral with or fixedly attached to head 122, and an unthreaded convex or substantially conical joining section 162 is provided in a particular embodiment between connecting portion 124 and shaft 160.

Cap member 126, in the embodiment of FIG. 2, includes a hole 166 and cut-outs 168 generally on either side of hole 166. In that embodiment, cut-outs 168 define lower side surfaces 170 that in a particular embodiment are sloped outwardly from the middle of cap member 126 toward the bottom of cap member 126, e.g. sloped surfaces 170 face generally away from hole 166. Cut-outs or undercuts 168 form fingers or flanges 171 that extend on either side of hole 166, and form an extension 172 that may be generally trapezoidal or tenon-shaped with surfaces 170. Hole 166 is threaded in one embodiment, and is positioned and of a diameter such that an extension of hole 166 would not intersect cut-outs 168. Use of the terms “cut-outs” and “undercuts” herein is not intended to limit a method of making cap member 126 to one in which material is cut out of a workpiece to form the surfaces disclosed in the above embodiment. Any appropriate known method of manufacturing can be used to make cap member 126. The illustrated embodiment of lock member 128 is similar to the embodiment of lock member 28 disclosed above insofar as lock member 128 is depicted as a type of set screw.

Referring to FIG. 6, another embodiment of cap member 126′ and head 122′ is shown. In most respects, cap member 126′ and head 122′ are the same as the above-discussed embodiments of cap member 126 and head 122, and for clarity's and brevity's sake similar parts are not further discussed here. The principal difference between these embodiments is the square shape of cut-outs 168′ of cap member 126′, having inner surfaces 170′ and fingers or flanges 171′ to form substantially an inverted T-shape, and of the opening atop each branch 130, 132. Accordingly, the embodiment of head 122′ shown in FIG. 6 includes surfaces 142′, 144′ that are substantially perpendicular to surfaces 146a, 146b and 148 (and/or substantially perpendicular to channel 134), and surfaces 143′ and 145′ that are substantially parallel to surfaces 146a, 146b and 148. The embodiment of cap member 126′ includes cut-outs 168′ with internal surfaces 170′ that are substantially perpendicular to base 164, and flanges 171′ that are substantially parallel to base 164. In addition to substantially tenon-and-mortise and T-shaped configurations, other configurations for cap member 126 and head 122 can be used.

Use of implant 120 is substantially similar to that described above with respect to implant 20. Once connecting member 124 is connected to bone and at least a portion of an elongated member is in channel 134, cap member 126 is placed on head 122. In the illustrated embodiment of implant 120, cap member 126 slides onto head 122 substantially in a direction perpendicular to channel 134 and to the longitudinal axis of connecting portion 124. In other embodiments, cap member 126 and head 122 can be configured so that cap member 126 moves onto head 122 in other directions that are non-parallel to or substantially not along the axis of channel 134. Cap member 126 is positioned so that surfaces 170 are adjacent surfaces 142, 144 of extensions 140a, 140b and fingers 171 are adjacent generally horizontal surfaces 148. Cap member 126 slides across head 122, with surfaces 170 and surfaces 142, 144 sliding along each other. Cap member 126 is advanced across head 122 (e.g. in the direction of the arrow in FIG. 2), in the illustrated embodiment, until surfaces 170 of cap member 126 are adjacent to or abut surfaces 142, 144 of extensions 140a and 140b of both branches 130, 132.

When cap member 126 is in place as indicated, extensions 140a, 140b of branches 130, 132 and the lower part of cap member 126 form essentially mortise-and-tenon or dovetail joints on each side of head 122. With cap member 126 atop head 122 so as to close head 122, the elongated member (in channel 134) and/or implant 120 can be adjusted relative to each other or to the adjacent vertebrae. For example, implant 120 can be pushed, pulled or rotated along with its attached vertebra so as to compress, distract or rotate the vertebra with respect to other bones or tissue. As another example, the elongated member can be further bent or turned in channel 134 to a better position or configuration, while cap member 126 retains the elongated member within channel 134. In embodiments in which implant 120 is a pivotable or multi-axial implant, the head of such an implant can be pivoted with respect to its bone connecting portion and/or the elongated member in the head for better support.

Once any such adjustments are made, lock member 128 is engaged to lock the elongated member to head 122 of implant 120. In the illustrated embodiment, in which lock member 128 is a set screw, the set screw can be threaded into hole 166 of cap member 126 prior to or after placement of cap member 126 on head 122. In either case, when implant 120 and its associated vertebra is positioned as the surgeon desires with respect to other vertebrae, implants and/or the elongated member, the set screw is tightened into contact with the elongated member to the desired degree. Lock member 128 exerts force on the elongated member, which presses back on the lock member and the threads of hole 166, and surfaces 170 of cap member 26 are forced against surfaces 142, 144 of head 122. The elongated member and head 122 are thus locked with respect to each other.

The embodiment shown in FIG. 2A is used in essentially the same fashion as the embodiment shown in FIG. 2. When cap member 126′ is slid onto head 122′, a square joint is formed in which flanges 171′ are adjacent surfaces 142′, 143′, 144′ and 145′. Insertion of a lock member, such as lock member 128, through cap member 126′ to exert force on an elongated member locks the construct.

Referring now generally to FIGS. 3-4, there is shown an embodiment of an implant 220 similar to that of implants 20 and 120. Implant 220 includes a head portion 222, a bone connecting portion 224, a cap member 226, and a lock member 228. In general, the illustrated embodiments of connecting portion 224 and lock member 228 are essentially identical to the embodiments of connecting portion 24, 124 and lock member 128 disclosed above.

Head portion 222 of implant 220 includes two branches 230, 232 that at least partially define a channel 234 for accommodating an elongated member such as an orthopedic rod R. Channel 234 has a longitudinal axis substantially along or parallel to which rod R can lie. In this embodiment branch 232 is shorter than branch 230, and channel 234 is substantially L-shaped. In other embodiments, branches 230 and 232 could be approximately the same height or somewhat closer in height, and channel 234 could be somewhat J-shaped. The illustrated embodiment of head portion 222 shows branches 230, 232 extending generally upward or away from connecting portion 224, but in other embodiments branches 230, 232 could be otherwise oriented, e.g. oblique to an axis of connecting portion 224 or substantially perpendicular to an axis of connecting portion 224, making a channel that opens somewhat or entirely to the side of such an axis.

Branch 230 has a groove 240 substantially parallel to a longitudinal axis of channel 234 and at or near the top of branch 230 in the illustrated embodiment. A sloped or substantially horizontal surface 242 may also be placed at or near the top of branch 230 and adjacent groove 240. Branch 232 includes a groove 244, which in this embodiment is also substantially parallel to a longitudinal axis of channel 234 and at or near an end of branch 232. If desired, a sloped surface similar to surface 242 may be placed on an outside of branch 232 adjacent groove 244. On an outside surface of branch 230 an indentation 250 may be provided adjacent groove 242 for accommodating a part of cap member 226, a holding or manipulating tool (not shown), or other apparatus. As already noted, connecting portion 224 is essentially identical to the embodiment of connecting portion 24 above, and thus includes a threaded shaft 260 that is integral with or fixedly attached to head 222, and an unthreaded convex or substantially conical joining section 262 is provided in a particular embodiment between connecting portion 224 and shaft 260. In other embodiments, a boss or stop may be provided on one or both of head 222 and cap member 226, which may limit or inhibit movement of cap member 226 parallel to elongated member R.

Cap member 226, in the illustrated embodiment, includes a base 264, a hole 266 through base 264, and extension portions or legs 268a, 268b generally perpendicular to base 264 and on either side of hole 266. In that embodiment, legs 268a, 268b extend along substantially the entire width of base 264, although in other embodiments legs 268a, 268b could have one or more gaps. Leg 268a is shorter than leg 268b in the illustrated embodiment, and in a particular embodiment the difference in height between legs 268a and 268b corresponds somewhat to the height difference between branches 230 and 232 of head 222. Leg 268a includes an internal surface 270a that may be substantially horizontal or may slope outwardly as it moves toward the edge of cap member 226, and may form a tongue 271a. Leg 268a may also have an external surface 272 that is curved or sloped so that it can relatively easily pass sloped surface 242 of branch 230. The illustrated embodiment of leg 268b also includes an internal surface 270b that is horizontal or slopes outwardly as it moves toward the edge of cap member 226, and may form a tongue 271b. Hole 266 is threaded in one embodiment, and is positioned and of a diameter such that an extension of hole 266 would not intersect legs 268a, 268b. In other embodiments, one or both of surface 270a and tongue 271a may include one or more surfaces that are non-linear individually or together, which may also limit or inhibit motion of cap member 226 parallel to elongated member R. As previously discussed, the illustrated embodiment of lock member 228 is similar to the embodiment of lock member 128 disclosed above.

Use of implant 220 is substantially similar to that described above with respect to implant 20. Once connecting member 224 is connected to bone and at least a portion of an elongated member is in channel 234, cap member 226 is placed on head 222. In the illustrated embodiment of implant 220, leg 268b of cap member 226 is connected to branch 232 of head 222 so that surface 270b is adjacent groove 244. Leg 268b may be inserted directly into groove 244 from the side (e.g. a direction substantially perpendicular to channel 234), or in surgical situations in which implant 220 is not too close to an adjacent implant, may be slid into groove 244 in a direction essentially parallel to channel 234. Cap 226 can be pivoted around the connection between leg 268b and branch 232 so that leg 268a is adjacent branch 230. In a particular embodiment, cap 226 moves toward head 222 in a direction that is substantially perpendicular to the longitudinal axis of channel 234. In other embodiments, cap member 226 and head 222 can be configured so that cap member 226 moves onto head 222 in other directions that are non-parallel to or substantially not along the axis of channel 234. In a particular embodiment, leg 268a can abut sloped surface 242 of branch 230, and is then forced past surface 242 so that surface 270a snaps into or is otherwise adjacent groove 240. In some surgical situations, the rotation of cap member 226 and its connections to head 222 can be used to force an elongated member into, or further into, channel 234. Adjustments may be made to cap member 226 as may be desired.

When cap member 226 is in place as indicated, tongues 271a, 271b of branches 230, 232 and grooves 240, 242 of cap member 226 fit in essentially tongue-and-groove fashion on each side of head 222, and the sloped surfaces 270a, 270b prevent cap member 226 from being lifted off of head 222 generally along a longitudinal axis of connecting member 224. With cap member 226 atop head 222 so as to close head 222, the elongated member R (in channel 234) and/or implant 220 can be adjusted relative to each other or to the adjacent vertebrae. For example, implant 220 can be pushed, pulled or rotated along with its attached vertebra so as to compress, distract or rotate the vertebra with respect to other bones or tissue. As another example, the elongated member can be further bent or turned in channel 234 to a better position or configuration, while cap member 226 retains the elongated member within channel 234. In embodiments in which implant 220 is a pivotable or multi-axial implant, the head of such an implant can be pivoted with respect to its bone connecting portion and/or the elongated member in the head for better support.

Once any such adjustments are made, lock member 228 is engaged to lock the elongated member to head 222 of implant 220. In the illustrated embodiment, in which lock member 228 is a set screw, the set screw can be threaded into hole 266 of cap member 226 prior to or after placement of cap member 226 on head 222. In either case, when implant 220 and its associated vertebra is positioned as the surgeon desires with respect to other vertebrae, implants and/or the elongated member, lock member 228 is tightened into contact with the elongated member to the desired degree. Lock member 228 exerts force on the elongated member, which presses back on lock member 228 and the threads of hole 266, and legs 268a, 268b of cap member 226 are forced against branches 230, 232 of head 222. The elongated member and head 222 are thus locked with respect to each other.

The devices of the present invention are preferably constructed of sturdy bio-compatible materials, such as stainless steel, titanium, certain plastics, or other known materials. Aspects disclosed with respect to one embodiment may be used or included in or with respect to other embodiments.

While the disclosure has been illustrated and described in detail in the drawings and foregoing description, the same is to be considered as illustrative and not restrictive in character, it being understood that only embodiments have been shown and described and that all changes and modifications that come within the spirit of the disclosure are desired to be protected.

What is claimed is:

1. An apparatus comprising:
a bone connection member with a head having two branches and a channel between said branches for accommodating an elongated member;
a cap member connected to said head and substantially closing at least part of said channel, said cap member having a hole therethrough and at least one extension portion, said cap member being movable with respect to said head in a direction generally perpendicular to said channel when said cap member is connected to said bone connection member;
a lock member connected to said cap member and extending through said hole and into said channel so as to lock the elongated member with respect to said head and said cap member with respect to said head.

2. The apparatus of claim 1, wherein said extension portion of said cap member includes a sloped surface and at least one of said branches includes an extension with a sloped surface, and said sloped surfaces are adjacent when said cap member is connected to said bone connection member.

3. The apparatus of claim 1, wherein said extension portion of said cap member includes a flange substantially perpendicular to said hole and at least one of said branches includes an extension with a surface in a plane substantially perpendicular to said channel, and said flange is adjacent said surface of said at least one of said branches when said cap member is connected to said bone connection member.

4. The apparatus of claim 1, wherein said cap member includes a base portion through which said hole extends and two extension portions, each said extension portion having a sloped surface generally facing said hole and sloping inward to form a mortise; and
wherein at least one of said branches includes a projection having lateral sloped surfaces to form a tenon, said sloped surfaces of said projection being adjacent said sloped surfaces of said extension portions when said cap member is connected to said bone connection member.

5. The apparatus of claim 1, wherein said extension portion of said cap member includes a first sloped surface on one side and a second sloped surface on a second side, said sloped surfaces generally facing away from said hole and sloping outward so that said extension portion is generally tenon-shaped; and
wherein a first of said branches includes at least one projection having a lateral sloped surface and a second of said branches includes at least one projection having a lateral sloped surface, said sloped surfaces of said projections being adjacent said sloped surfaces of said extension portion when said cap member is connected to said bone connection member.

6. The apparatus of claim 1, wherein said cap member includes a base portion through which said hole extends, two extension portions, each said extension portion having an end distal from said base portion and a tongue adjacent said end; and
wherein a first of said branches includes a groove for accommodating said tongue of a first of said extension portions when said cap member is connected to said bone connection member, and a second of said branches includes a groove for accommodating said tongue of a second of said extension portions when said cap member is connected to said bone connection member, and said cap member is pivotable around said tongue of said first extension portion in said groove of said first branch.

7. The apparatus of claim 6, wherein said first branch is longer than said second branch.

8. A method comprising:
providing an implant having a bone connection member and a cap member, said bone connection member having a channel for accommodating an elongated member substantially along a channel axis;
connecting said bone connection member to a bone; and
placing said cap member into connection with said bone connection member, said placing being in a direction non-parallel to said channel axis.

9. The method of claim 8, further comprising inserting an elongated member at least partially in said channel.

10. The method of claim 9, wherein said inserting step occurs before said placing step.

11. The method of claim 98, further comprising locking said cap member, said bone connection member and said elongated member together.

12. The method of claim 8, wherein said placing step includes sliding said cap member into connection with said bone connection member.

13. The method of claim 8, wherein said placing step includes rotating said cap member into connection with said bone connection member.

14. The method of claim 8, wherein said placing step includes moving said cap member in a direction substantially perpendicular to said channel axis.

15. An apparatus comprising:
a bone connection member with a head having two branches and a channel between said branches for accommodating an elongated member, at least one of said branches having at least one projection;
a cap member having a base portion with a hole therethrough, a first extension portion on a first side of said hole, and a second extension portion on a second side of said hole, said cap member being slidably connected to said head so that said extension portions of said cap member are adjacent said at least one projection of said head, said cap member substantially closing at least part of said channel, said cap member being slidable with respect to said head in a direction non-parallel to said channel;
a lock member connected to said cap member and extending through said hole and into said channel so as to lock the elongated member with respect to said head and said cap member with respect to said head.

16. The apparatus of claim 15, wherein said projection has two ends each having a sloped surface, said sloped surfaces facing generally along said channel axis.

17. The apparatus of claim 16, wherein said extension portions of said cap member each have sloped surfaces that are adjacent said sloped surfaces of said projection when said cap member is connected to said head.

18. The apparatus of claim 17, wherein said sloped surfaces of said extensions form a first angle with respect to said base portion of said cap member, and said projection includes an upper surface and said sloped surfaces of said projection form a second angle with respect to said upper surface of said projection, and wherein said first and second angles are approximately equal.

19. An apparatus comprising:
a bone connection member with a head having two branches and a channel between said branches for accommodating an elongated member, a first of said branches having a projection with an undercut surface, and a second of said branches having a projection with an undercut surface, said undercut surfaces facing generally along said channel;
a cap member connected to said head and substantially closing at least part of said channel, said cap member having a hole therethrough and at least one extension portion, said extension portion being substantially between said projections of said branches, and said cap member being movable with respect to said head in a direction non-parallel to said channel;
a lock member connected to said cap member and extending into said channel so as to lock the elongated member with respect to said head and said cap member with respect to said head.

20. The apparatus of claim 19, wherein said cap member has a first side and a second side, and said at least one extension has an undercut surface adjacent said first side and an undercut surface adjacent said second side, said undercut surfaces of said cap member being adjacent said undercut surfaces of said projections when said cap member is connected to said head.

21. The apparatus of claim 20, wherein said undercut surfaces of said cap member form a first angle, and said undercut surfaces of said projections form a second angle with respect to an upper surface of their respective branches, and wherein said first and second angles are approximately equal.

22. An apparatus comprising:
a bone connection member with a head having first and second branches and a channel between said branches for accommodating an elongated member;
a cap member connected to said head and substantially closing at least part of said channel, said cap member having a base portion, a first extension portion, a second extension portion, and a hole through said base portion between said extension portions, said first extension portion being longer than said second extension portion, wherein said first extension portion is connected to said first branch and said cap member is rotatable around said first branch to connect said second extension portion to said second branch;
a lock member connected to said cap member and extending through said hole and into said channel so as to lock the elongated member with respect to said head and said cap member with respect to said head.
